(19) United States
(12) Patent Application Publication
(10) Pub. No.: US 2007/0092401 A1
(43) Pub. Date: Apr. 26, 2007

(54) Devices and methods for sample collection and analysis

(76) Inventors: Feier Liao, Hangzhou (CN); Shaomin Sun, Hangzhou (CN)

Correspondence Address: AZURE INSTITUTE;INTELLECTUAL PROPERTY DEPT., 4108 SORRENTO VALLEY BOULEVARD, SAN DIEGO, CA, 92124, US

(21) Appl. No.: 11/259,735

(22) Filed: Oct. 26, 2005

(51) Int. Cl. G01N 33/00

(52) U.S. Cl. 422/58; 422/68.1

(57) ABSTRACT

The present invention provides devices, methods, and kits for the collection of a solid or semi-solid sample and analysis for the presence, absence, or quantity of an analyte. The invention provides an assay device having a housing containing a test element, a results window, and a docking area for receiving and engaging a sample collection slide. The docking area has a sample receiving orifice with one or more fluid transfer structures. In one embodiment the collection slide and device can be used to detect the presence of fecal occult blood (human hemoglobin) in a stool sample. Many other embodiments are described herein.

### FIELD OF THE INVENTION

The present invention is directed to devices for the collection of solid or semi-solid biological samples, and their analysis for the presence of analytes.

### BACKGROUND OF THE INVENTION

The following Background of the Invention is intended to aid the reader in understanding the invention and is not admitted to be prior art.

The detection of occult blood in stool samples is a preliminary method of detecting colon cancer. Traditional methods that detect hemoglobin in a stool sample, such as Guaiac-based chemical methods, are hampered by their inability to distinguish between dietary-derived hemoglobin (i.e. from meat in the diet) and human hemoglobin, which leads to a large number of false-positive test results. To over-come this difficulty, immunoassays specific for human hemoglobin (hHb) have been developed. The antibodies used in these assays are able to distinguish between hemoglobin derived from a human and that from another animal.

The collection and analysis of occult blood samples presents the problem of the unpleasantness of sample collection and analysis. Presently available devices fail to adequately solve these problems. Therefore, there is a clear and persistent need for a device that reduces the interaction of both the patient and the test operator with the sample while at the same time accurately detecting the presence of hHb in the sample.

### SUMMARY OF THE INVENTION

The present invention provides devices, methods, and kits for collection of a biological sample, and the detection of an analyte in the sample. In one embodiment, the biological sample is a stool sample and the analyte is hemoglobin. The sample is collected on a collection slide that can be used with the device. The device contains a test element, such as a test strip, that has reagents for detecting the analyte. The device also contains a docking area for receiving the collection slide. The docking area contains a sample receiving orifice with one or more fluid transfer structures (e.g., a crossbar extending across the orifice) that facilitate the transfer of fluid from a sample collection card to the device. The fluid transfer structure facilitates the movement of fluid from the card to the device by providing a surface for fluid to adhere to and travel down to the test element.

In one aspect, the present invention provides a device for detecting an analyte in a sample. The device has a housing that contains a test element, and a docking area for receiving and engaging an external collection slide. The docking area has a sample receiving orifice that has one or more fluid transfer structures within the circumference of the sample receiving orifice. A results window for observing a test result is also provided on the housing.

In one embodiment, the sample receiving orifice is a well in the housing of the device. In another embodiment, the fluid transfer structure is a crossbar, which can project below, level with, or above the plane of the docking area. The crossbar is in fluid communication with an engaged collection slide. In another embodiment, the docking area has one or more projections for securing the external sample collection slide in position above the sample receiving orifice. The one or more projections can be snap locks. In another embodiment, the docking area is a depression in the housing. The depression can be at least partially circumscribed by a raised area of the housing.

In another embodiment, the test element is made of a bibulous matrix, which has a sample application zone (in fluid communication with the one or more fluid transfer structures), a reagent zone (containing reagents for conducting an assay) and a detection zone. The detection zone contains a test line, for visually detecting the presence or absence of the analyte at the test line. The test line can also contain a specific binding molecule, for the analyte, immobilized on the matrix. In some embodiments, the specific binding molecule is an antibody. In other embodiments, the specific binding molecule on the test line binds to human hemoglobin. In still another embodiment, the reagent zone contains labeled specific binding molecule for the analyte.

In another aspect, the present invention provides methods of detecting the presence or absence of an analyte in a sample contained in a sample collection slide. The methods involve placing a collection slide containing the sample into a docking area of a device for detecting analyte in the sample as described herein. In one embodiment, the collection slide has a first water resistant card with an eluent orifice, a second water resistant card hingeably connected to the first card and having a solvent orifice. The collection slide can have both an open position and a closed position, and a sample collection surface is present between the solvent and eluent orifices, when the collection slide is in the closed position. The method further involves applying an extraction buffer to the solvent orifice of the collection slide, allowing the extraction buffer to pass through the sample area and through the sample receiving orifice and test element, and observing a test result in the results window.

In one embodiment, the test element is a bibulous matrix having a sample application zone in fluid communication with the one or more fluid transfer structures, a reagent zone, including reagents for conducting an assay, and a detection zone having a test line for detecting the presence or absence of the analyte. The test line can also include specific binding molecules for the analyte. In another embodiment, the test line contains reagents for conducting a chemical test.

In another aspect, the present invention provides a kit for collecting a biological sample. The kit includes a test device of the present invention, a collection card and a sample collector, as described herein, provided in a package. In a further embodiment, the kit includes one or more bottles containing buffers. The buffers are for conducing an assay according to the instructions for use.

The summary of the invention described above is not limiting and other features and advantages of the invention will be apparent from the following detailed description, as well as from the claims.

### BRIEF DESCRIPTION OF THE DRAWINGS

FIG. 1 provides a perspective view of an embodiment of the invention, which includes a sample collection slide 110 and a test device 120 that engages the collection slide. Also shown is the sample collector 134 for applying the sample to the collection slide.

FIG. 2 provides an exploded view of the devices shown in FIG. 1.

FIGS. 3A-3C illustrate application of a sample to the collection slide. FIG. 3A illustrates an opened collection slide, showing a cover pad 218 and a collection pad 216. FIG. 3B illustrates application of the sample 310 to the collection pad. FIG. 3C illustrates a closed collection slide.

FIG. 4 illustrates a collection slide 110 engaging the docking area 126 of a test device.

FIG. 5 illustrates application of extraction buffer 512 to the solvent orifice 116 of an engaged collection slide.

FIG. 6 provides a cross-sectional view of a collection slide 110 engaged in a test device 120.

### DETAILED DESCRIPTION

In the following detailed description, reference is made to the accompanying drawings that form a part hereof, and in which is shown by way of illustration specific embodiments in which the invention may be practiced. It is understood that with reference to the present disclosure other embodiments may be utilized and structural changes may be made without departing from the scope of the present invention.

Collection Slide

The present invention provides collection slides for collecting a solid or semi-solid sample. In some embodiments the sample is a biological sample, such as a stool sample. The present invention also provides devices for detecting the presence of analytes in the sample, and methods for collecting the sample.

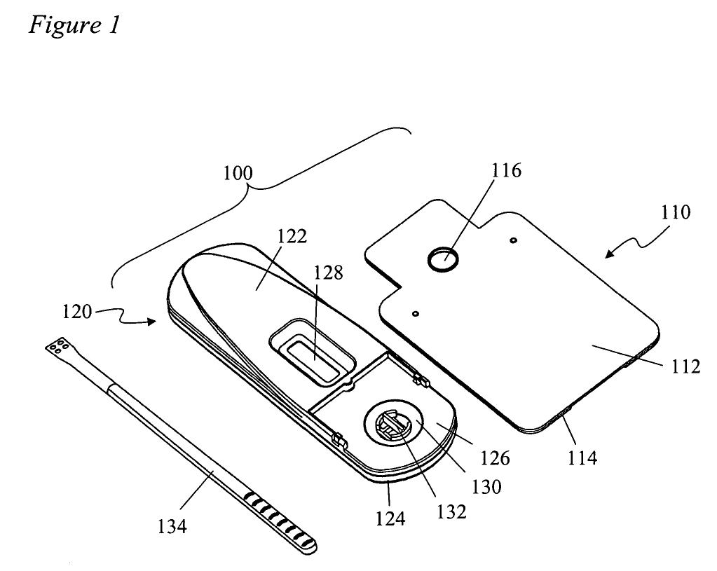

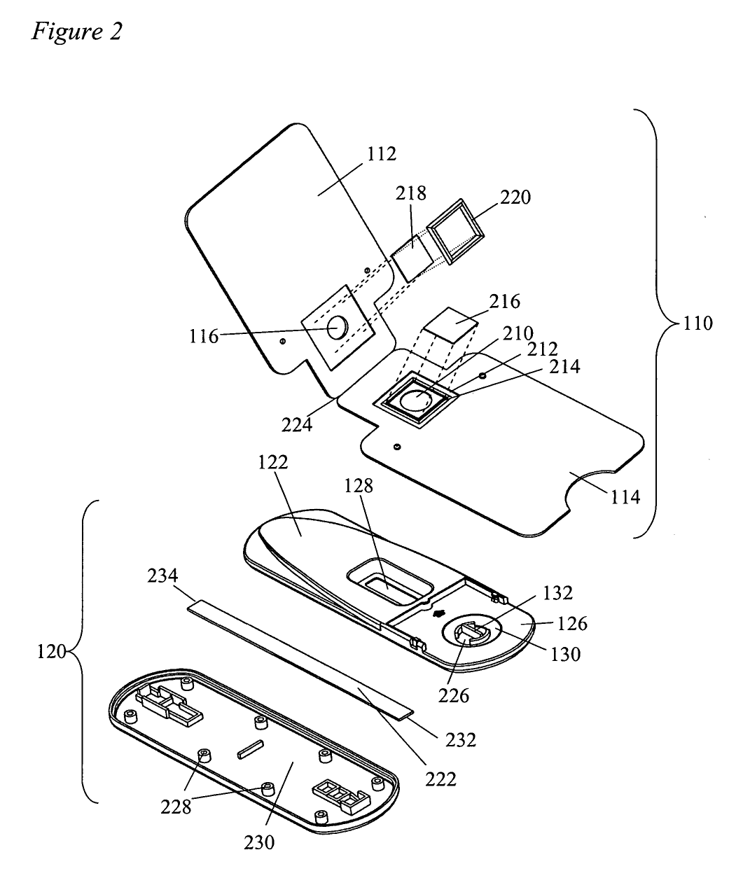

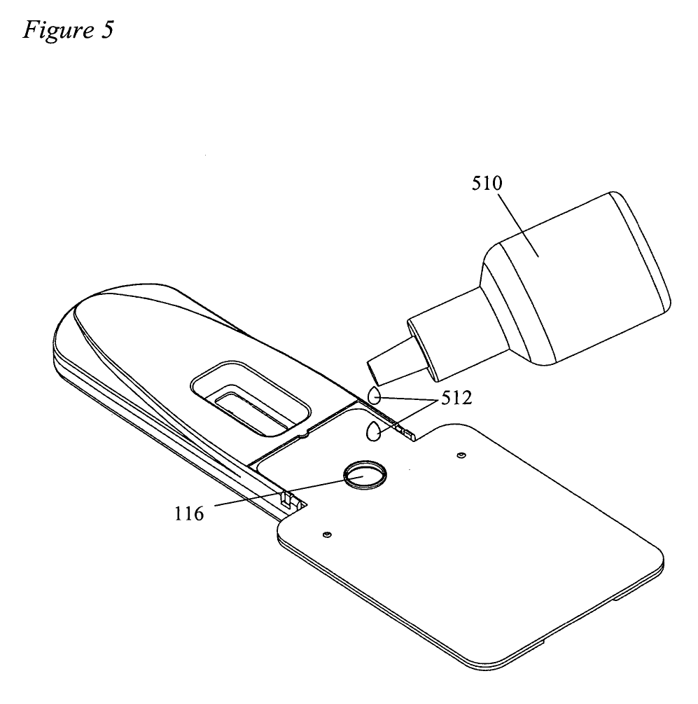

The test device of the present invention can be used with an external collection slide 110. With reference to FIGS. 1-5, the collection slide 110 has a first card 114 and a second card 112. The first and second cards may be made of any appropriate material. For example, the cards can be made of a resilient, water resistant or water-impermeable material, such as plastic, coated cardboard, metal or glass. In one example, the cards are hingeably connected to each other, for example by a hinge 224 (FIG. 2). By “hingeably connected” is meant that the two cards are connected to each other at their first ends and have free ends movable towards and away from each other by movement about the hinge. A wide variety of hinge connections may be advantageously used. In the example shown in the figures, the collection slide is manufactured of injection molded plastic and the two cards are connected by a living hinge, as depicted in FIG. 2. In other examples, the hinge can be one or more flaps of material that bind the two cards together and allow for one card to be folded onto the other card. In another example the cards are present as separate cards that can be secured together, for example by a locking mechanism. The second card has a buffer or solvent orifice 116, through which an extraction buffer 510, 512 can be applied to a collected sample (FIGS. 1 and 5).

The collection slide has an open position and a closed position (compare FIGS. 1 and 2). As illustrated in FIG. 2, the first card has an eluent orifice 210 and the second card has a solvent orifice 116. The solvent and eluent orifices are positioned on the cards so that when the collection slide is in the closed position, the two orifices are in alignment. By the orifices being “aligned” or “in alignment” is meant that a liquid applied to the solvent orifice in the second (or top) card in sufficient quantity will pass through the sample collection area and through the eluent orifice.

Referring to FIG. 2, a cover pad 218 is present on the inner surface of the second card and overlaying the buffer orifice 116. The cover pad and sample collection pad can be made of any suitable material that retains sample and allows the passage of fluid. Examples of materials suitable for the cover pad and/or sample collection pad are polyester mesh, fibrous or bibulous materials, paper or paper-based materials, synthetic fabrics, meshes and wools, coated or supported papers, polyesters, nylon membranes, nitrocellulose, glass wool, treated paper, absorbent paper, or a material made of a cellulose base. In the example shown, the cover pad 218 is circumscribed by a gasket 220. With reference to the present disclosure the person of ordinary skill in the art will realize many other materials suitable for the cover pad and/or sample application pad.

On the first card is present an eluent orifice 210, which is overlaid with a sample collection pad 216. The sample collection pad 216 can be made of any suitable material that retains sample and allows for the passage of fluid. In various examples the sample collection pad 216 is made of the same types of materials as the cover pad. The sample collection pad can be circumscribed by ridge 214 and groove 212, or by a series of ridges and grooves. The cover pad and the collection pad can be made of any suitable material that retains sample and allows for the passage of fluid. Examples are provided above with respect to material for the cover pad. The material should also have sufficient resiliency to withstand the mechanical pressure of the sample application. Preferably, the material does not deteriorate or tear when wet.

Common difficulties with stool sample collection include that patients tend to over-apply sample to collection slides, which can cause interference when the assay is an immunoassay. Collection slides used with a test device of the present invention can desirably limit the amount of sample that can be applied to the slide while requiring no direct sample manipulation by the technician conducting the test. Using such a slide, the amount of sample collected is limited to the sample collection area since the cover pad and sample collection pad are circumscribed by the sealing structures (e.g., a gasket and groove) when the slide is in the closed position. When the collection slide is moved to the closed position, the interaction of the sealing structures (e.g., the interaction of the gasket with the groove and ridge) separates the sample within the sample application area from sample applied outside the sample area. After the sample has been applied to the sample collection area, the collection slide is closed and retained in a locked position, thereby limiting the volume of sample retained within the sample area, because excess sample is squeezed out as the two cards are pressed together. The sealing structures can also be structures other than a gasket, ridge, and groove. For example, the structures can be a pressure sensitive adhesive or a wax bead (or beads) present on or around the sample collection pad and/or cover pad, which seal the sample collection pad when the two cards are closed and pressed together. The “seal” does not have to be a tight seal, just that it generally impedes the passage of sample into or out of the sample collection area when the collection slide is in the closed position. With reference to this disclosure the person of ordinary skill will realize many other structures that will find use in other examples of the collection card.

The cover pad and/or collection pad can be treated with reagents that improve the flow of aqueous liquids through them. Additionally, these treatments also improve the elution of the analyte of interest from the dried sample within the sample area. In one example the pads are treated with surfactants to inhibit proteins from sticking to the pads and to promote protein solubilization. A wide variety of commonly used anionic and non-ionic surfactants may be advantageously used in various concentrations. Some cationic and amphoteric surfactants may also find use in the present invention. Some examples of surfactants that may be used to treat the pads include, but are not limited to, the polyoxyethylene fatty ethers derived from lauryl, cetyl, stearyl and oleyl alcohols (e.g., the BRIJ® (ICI US, Inc.) series of surfactants). Other useful surfactants include octyl phenol ethoxylate surfactants (e.g., polyethyrene glycol mono-p-iso-octylphenyl ether and other Triton® (Rohm & Haas, Philadelphia, Pa.) series surfactants), polyoxyethylene derivatives of sorbitan esters (e.g., the Tween® (ICI Americas, Inc.) series of surfactants) and block copolymers based on ethylene oxide and propylene oxide and represented by HO(C₂H₄O)ₐ(C₃H₆O)_{b}(C₂H₄O)ₐH (e.g., the Pluronic® (BASF) series of surfactants). With reference to the present disclosure, a surfactant can be conveniently chosen using known surfactant selection techniques, such as by using a commercially available surfactant tool kit, for example, the Reagent Developer's Surfactant Took Kit (Pragmatics, Inc., Elkhart, Ind.), or a similar kit. These kits provide a convenient method of testing a large number of surfactants on a specific application, in order to optimize protein extraction and flow-through.

In some embodiments the pads are treated with a buffer containing a component that improves analyte stability. Buffers can also condition the sample to promote optimal binding between the analyte and the specific binding reagents (e.g., antibodies or antibody fragments), which can be utilized in the assay. This can be performed, for example, by adjusting the pH of the analyte. Buffers having these useful qualities include, but are not limited to, Tris(hydroxymethyl)aminomethane buffer, phosphate buffer, borate buffer, tartrate buffer and phthalate buffer.

The cover pad and/or sample application pad can also be treated with one or more polymers, which can also have the property of improving analyte stability and elution. Polymers sometimes used in protein purification can be useful for this purpose. Examples of useful polymers include, but are not limited to, polyvinylpyrrolidone (PVP), poly(methylvinylether-co-maleic anhydride, polyethylene oxide (PEO), polyelthylene glycol (PEG), copolymers of methyl vinyl ether and maleic anhydride (e.g., poly(methylvinylether-co-maleic anhydride), polyvinylalcohol (PVA), vinylpyrrolidone/vinylacetate, bony fish gelatin (from fish of the class Osteichthyes), crosslinked polyacrylic acid polymer, hydroxypropylcellulose (HPC), sodium carboxymethylcelluose (CMC), sodium polystyrenesulfonate, sodium carageenin, acrylic latex, and hydroxyethylcellulose (HEC)). These polymers are commercially available (e.g., from Pragmatics, Inc., Elkhart, Ind.), and are conveniently formulated in a polymer tool kit. They can therefore be used systematically to determine the advantages of particular polymers in particular applications.

To improve analyte extraction, the pads may also be treated with a non-specific protein, which functions as a blocking agent. Any protein may be used for this purpose including, but are not limited to, bovine serum albumin, egg white albumin, and casein.

The cover pad and sample application pad can also be treated with a preservative to increase the shelf-life of the collection slide. A “preservative” is a naturally or synthetically produced chemical added to inhibit microbial growth or undesirable chemical changes. Any preservative can be used that provides the preserving effect and does not interfere with the assay. Examples of useful preservatives include, but are not limited to, 5-chloro-2-methyl-isothiazol-3-one (e.g. ProClin® 300 (Supelco, Inc., Bellefonte, Pa.) and sodium azide. With reference to the present disclosure the person of ordinary skill will realize many other preservatives that will find use in the present invention.

The cover pad and collection pad form the top and bottom walls of the sample collection area, and serve to eliminate excess sample from the sample collection area. When the structures on the cards are a gasket, ridge, or groove, they can also be situated on the opposite cards as those described above.

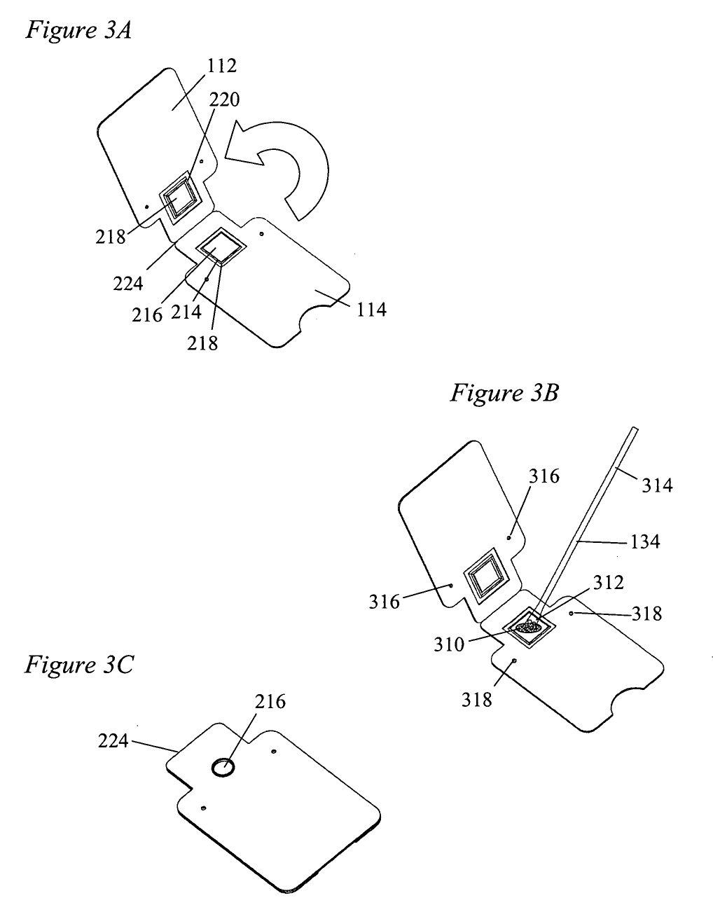

In certain sample collection slides, one of the cards of the collection slide is provided with structures for securing the first and second cards in a closed position. In one example short pins 316 (FIG. 3B) are present on the interior surface of one card. The opposite card is provided with holes 318 that mate with the pins. When the collection slide is closed, the pins are inserted into the holes and lodged with sufficient resistance to hold the collection slide in a closed or “locked” position. In one example this action may advantageously cause a snapping noise, alerting the patient that the collection slide has been properly closed. Other methods of securing the collection slide in a closed position can also be incorporated into the slide. For example, a clip that fits over the outside of the two cards and holds them together could be used in one example, or snaps present on the inner surfaces of the two cards can be used in another example. With reference to the present disclosure the person of ordinary skill will realize other structures for retaining the collection slide in the closed position.

Sample Collector

The present invention also provides a sample collector 134, such as the embodiment shown in FIG. 1. The sample collector has a handle 314 (FIG. 3B) and a spatula 312 for moving the sample. In one embodiment the spatula is perforated with a plurality of holes, which reduces the liquid content of the sample, and also serves to reduce application of excess sample to the sample collection pad. In various embodiments the spatula portion of the device is perforated with 2, 3, 4, 5, 6, 7, 8, 9, 10, 11, 12, or more holes. The spatula portion of the collector can be generally flat, or can have a concave or curved (spoon-like) shape. This device can be made of any suitable material (e.g., plastic). In one embodiment, the spatula portion of the device is made of a soft plastic, and the handle is made of a harder plastic. This will enable the spatula to bend when sample is applied to the sample collection pad and lay on the pad. The perforations in the spatula portion will also act as an aid in applying an even sample to the pad.

Methods of Collection

In another aspect the present invention provides methods of collecting a sample. In one embodiment the sample is a stool sample. One embodiment of the method of sample collection and operation of the collection slide and assay device is illustrated in FIGS. 3A-3C. Referring to FIG. 3A, the patient opens the collection slide to expose the inner surfaces of the first and second slides, revealing the cover pad and sample collection pad. A small amount of stool sample is applied to the sample collection pad 216. The collection slide is then closed (FIG. 3C). The present collection slide eliminates excess sample by providing a sample collection area, with a design such that only sample in the sample collection area will be incorporated into the assay. When the collection slide is closed, a structure the first card engages a structure second card, forming a wall that circumscribes the sample collection area. In one embodiment the structure on one card is a gasket, and the structure on the opposite card is a groove and a ridge. When the collection slide is in the closed position, the solvent or buffer orifice, the sample area, and the eluent orifice are all vertically aligned. In this position, when buffer is applied to the buffer orifice, it flows through the cover pad and into the sample collection area, and then out of the eluent orifice, thereby rinsing the sample in the process and solubilizing analyte of interest contained in the sample. Additionally, the buffer dilutes the sample and conditions it for optimal binding of analyte by the specific binding reagents on the test element. After passing through the eluent orifice, the liquefied sample is then passed along the fluid transfer structures of the device and through the sample receiving orifice, and to the test element of the device.

Human hemoglobin breaks down rapidly when left in a wet sample. To prevent analyte degradation, the methods can incorporate the step of drying the sample. This step can involve leaving the collection card exposed to air for a certain period of time to allow it to air dry, or drying the sample in an oven at 45° C. The step can also involve placing the closed collection slide into a container containing desiccant. The container can be a sealable pouch (e.g., a mailing pouch). After drying (or placing the collection slide in a sealable pouch containing a desiccant), the collection slide can be presented to a health care facility for analysis.

Assay Device

The present invention provides devices for detecting the presence of analytes in the sample, and methods for collecting the sample. The devices of the present invention can be used with collection slides for collecting a solid or semi-solid sample. In some embodiments of the present invention, the sample is a biological sample, such as a stool sample.

Referring to FIGS. 1 and 2, the assay device of this embodiment has a housing consisting of a top portion 122 and a bottom portion 124, which engage one another and lock together. The housing may be constructed of any suitable material such as, for example, plastics, pressed hardboard, metals, ceramics, polymers (e.g., polycarbonate, polypropylene, cycloolefins), and other materials. In the embodiment illustrated in the Figures, the housing is made of molded plastic. The top and bottom portions can engage one another by any convenient means, such as parts that snap together, glue, micro-welding, and other means. In the embodiment illustrated in FIG. 2, the top portion has a series of pins on the inner surface (not shown) which snap-fit snuggly into a corresponding series of raised rings 228 on the inner surface 230 of the bottom portion, thereby securing the top and bottom portions of the assay device in a locked position.

A docking area 126 for receiving and engaging a collection slide is located on the assay device. The collection slide may be “loaded” meaning that it contains a sample to be analyzed. The docking area may be of any shape, and can mate with a portion of the collection slide carrying the sample collection area. In one embodiment the docking area can receive and engage an external collection slide. An external collection slide is one that can be loaded separately from the assay device, and is not physically connected to the device at the time of sample loading. By “receiving and engaging” a collection slide is meant that the assay device and collection slide are placed into the “test position.” The “test position” is when the sample application pad and the fluid transfer structure(s) 132 are in liquid communication.

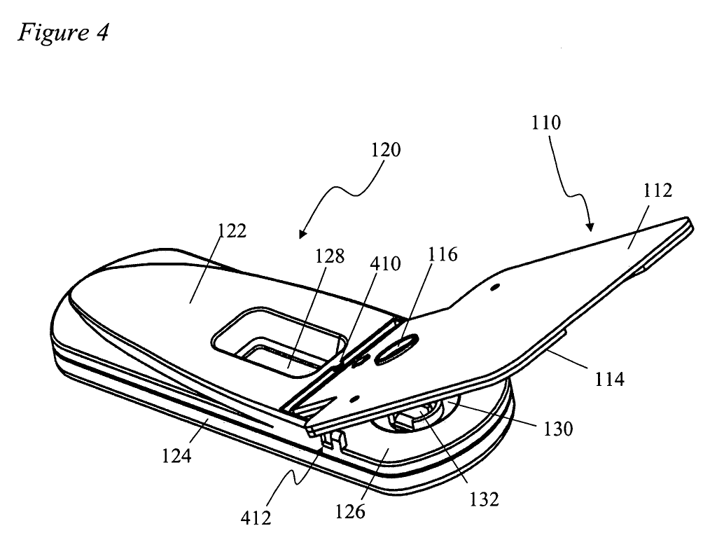

The docking area can also receive the collection slide in reversible fashion, meaning that the collection slide can be removed from the device after buffers are applied and sample eluted from the collection slide. As illustrated in FIG. 4, in this embodiment the collection slide is snapped into the docking area by fitting the hinged edge of the collection slide under a tang 241 (also see FIG. 2). The collection slide is then pressed down onto the docking area and snapped into a locked position under one or more projections 240. The projections hold the collection slide flush with the docking area. In other embodiments the collection slide is placed into the docking area of the assay device. In one embodiment the docking area can have a part that fits over the collection slide to hold it in place (e.g., an overhang that grips an end of the collection slide). When in place, the sample collection pad and the fluid transfer structure(s) are placed into fluid communication. The buffer orifice is exposed to receive buffer, and buffer applied to the buffer orifice passes through the sample collection pad and to the fluid transfer structure(s). In one embodiment the docking area is configured to receive the collection slide against an exterior surface of the assay device, so that the sample collection area and fluid transfer structure(s) are brought into liquid communication. The docking area can have projections for holding the collection slide securing in the test position.

In other embodiments the docking area can receive the collection slide into the interior of the device. For example, the collection slide can be slid into an opening in the housing of the device so that the sample application pad is placed into liquid communication with the fluid transfer structures. In another embodiment the sample transfer orifice is the only orifice in the assay device for receiving sample or assay fluids, and the sample and assay fluids both enter the device through the sample transfer orifice. “Assay fluids” refers to buffers or other reagents utilized during the assay. Thus, in these embodiments the sample transfer orifice is the sole orifice for receiving sample and fluids into the device.

As illustrated in FIG. 1, in one embodiment the docking area contains an indentation or well 130 having one or more fluid transfer structures 132 disposed therein. The fluid transfer structures can take any form that projects toward the collection slide and touches or nearly touches the exterior surface of the sample collection pad. For example, the fluid transfer structure(s) could be one or more raised bars attached to edge of the well. In another example, the fluid transfer structures can be a number of projections that extend towards the sample application pad of the collection slide. Any suitable number of projections can be used, such as one or 2 or 4 or 6 or 8 or 10 or 12, or 2-6 or 2-8 or 2-10 or 2-12 or 4-8.

In the embodiments shown in FIG. 2, the fluid transfer structure is present in the top portion of the housing, in the sample transfer orifice 226 and lies beneath, flush with, or slightly protruding through the plane of the docking area. In various examples, the fluid transfer structure can protrude 1 mm or 2 mm or 3 mm or 4 mm or 5 mm, or any suitable distance. The “plane” of the docking area is that spatial plane extending over the surface of the docking area and over the well.

In the embodiment illustrated, the fluid transfer structure is a cross bar spanning the diameter of the sample receiving orifice. In the case of more than one bar, the bars can be arranged parallel or at an angle to each other and intersecting (e.g., to form an “X” shape, or a grid, square, triangle, or a honeycomb pattern). In other embodiments the bars can connect any two points on the circumference of the sample receiving orifice. In other embodiments one or more vertically projecting prongs situated inside the well can also be used as fluid transfer structures. A straight or curved wall of any shape can be adapted to use as a fluid transfer structure.

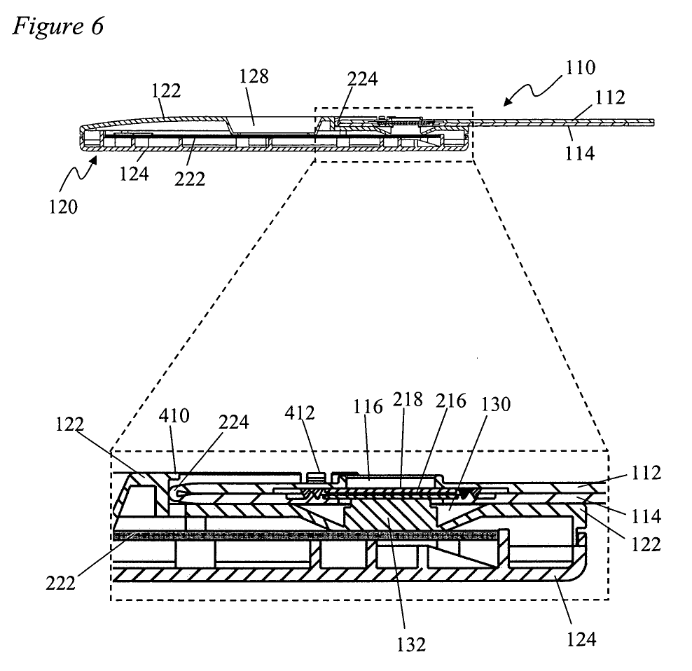

The fluid transfer structure facilitates the transfer of eluate emerging from the bottom of the sample collection pad to the test element. In one embodiment, when the collection slide is snapped into the docking area, the buffer orifice, cover pad, sample collection pad, eluent orifice, and fluid transfer structure(s) are all generally in vertical alignment with each other (FIG. 6). In this embodiment the fluid transfer structure projects towards, into, level with, or above the plane of the docking area, so that the fluid transfer structure and the outer surface of the sample collection pad are placed into fluid communication through the eluent orifice. By being in “fluid or liquid communication” is meant that fluid passing through the sample collection area and through the sample collection pad is passed to the fluid transfer structure. The sample collection pad and fluid transfer structure may make direct physical contact or be slightly apart from one another, but are retained in fluid communication.

Cohesion refers to the attraction of one water molecule to another resulting from hydrogen bonding. Adhesion is similar to cohesion except that adhesion involves the attraction of a water molecule to a non-water molecule, such as a surface. The fluid transfer structures in the present invention facilitate the movement of eluate collected on the exterior surface of the sample transfer pad by providing a surface for water molecules to be attracted to by adhesion. When the fluid transfer structure breaks the surface tension of the eluate, the eluate adheres to the surface of the fluid transfer structure. Adhesion of the eluate to the fluid transfer structure, in combination with the weight of the eluate, moves the eluate toward the sample pad of the test strip. Thus, the eluate flows to the test strip using adhesive forces along the fluid transfer structure(s). When the eluate contacts the sample pad of the test strip, the eluate is drawn into the sample pad by capillary action. Sufficient elution buffer is applied to the test card buffer orifice, that enough eluate is produced to flow, by capillary action, to the end of the test strip opposite the sample pad, and the test performed on the test strip can function properly.

As illustrated in FIGS. 2 and 6, a test element 222 is provided with the housing, and in this embodiment is contained within the housing. In this embodiment the test element is permanently situated within the housing of the device, meaning that it is not removable from the housing or inserted during the assay, but is an integral part of the assay device. Referring to FIG. 6, the fluid transfer structure is in fluid communication with the test element. In one embodiment, the test element is a bibulous test strip suitable for performing a lateral flow assay. A variety of test strips are suitable for use in the assay device. In one embodiment the test strips consist of a bibulous matrix, for example nitrocellulose, and/or other suitable materials. The matrix can have a sample loading zone, a reagent or label zone, and a detection zone. A variety of other test strips will also find use in the present invention. In some embodiments a sample loading zone is present at one end of the test strip for the application of sample to the test strip. The sample loading zone is the portion of the test strip in liquid communication with the transfer material. Reagents for conducting the assay or conditioning the sample can also be present at the sample loading zone, or they can be present in a separate reagent or label zone. These reagents can serve a variety of purposes, for example preparing the sample for optimal binding with a specific binding molecule, or improving the stability of an analyte of interest. By “conditioning” a sample is meant adjusting the characteristics of the sample to promote or improve the reaction that detects the presence of the analyte. For example, buffers may be included to adjust the pH of the sample. If the sample contains substances that compete for binding with a specific binding molecule used in the assay, a secondary blocking antibody can be included to bind the substance, or if enzymes that would degrade the specific binding molecules for the analyte are present in the sample, one or more enzyme inhibitors can be added to the reagent zone.

The sample loading zone is present at the upstream end 232 of the test strip. Towards the downstream end of the test strip 234 is the reagent zone, which is followed by a detection zone. The reagent zone can include reagents for conditioning the sample, reagents for labeling the analyte (e.g., specific binding molecules if the assay is a sandwich format immunoassay) or labeled analyte analogs (e.g., if the assay is a competitive format immunoassay). In some embodiments the reagent zone contains a labeled specific binding molecule for the analyte present on the matrix in a dried form, and which can be solubilized by sample fluid as it passes along the matrix. In one embodiment the specific binding molecule is an antibody or fragment thereof. In one embodiment the analyte is human hemoglobin (hHb), and the labeled specific binding molecule is an antibody that binds hHb. The antibody can be labeled by any suitable methods, for example, a metal sol, colored latex beads, and dyes. In some embodiments the sample loading zone and the reagent zone over-lap. In other embodiments there are present a series of reagent zones located on the test strip.

A “specific binding molecule” refers to a molecule that binds to a target analyte (e.g., human hemoglobin) and does not substantially bind to any other molecule present in the sample. In some embodiments a specific binding molecule can also bind to a molecule that correlates with or indicates the presence of an analyte of interest in a sample. By substantial binding is meant that binding occurs to an extent that will affect the result of an assay performed with the specific binding molecules, i.e., a less optimal or less accurate result will be obtained. A small amount of non-specific binding that may occur and that does not change the result of the assay is not considered substantial binding. In some embodiments the specific binding molecule can be an antibody or an antibody fragment (e.g., the Fab region of an antibody), an antigen, a receptor or fragment of a receptor that binds a ligand, or a member of a biotin-streptavidin pair or other type of binding pair.

The detection zone is the area of the test strip where the presence of the analyte is detected. In some embodiments the detection zone contains a test line for visually detecting the presence or absence of the analyte of interest at the test line. The test line can be of any shape, and need not be only a line. The test line can have a specific binding molecule for the analyte. When human hemoglobin is the analyte of interest, the specific binding molecule on the test line binds to hHb. In this embodiment the specific binding molecule binds to human Hb, and does not bind to hemoglobin that might be present from the diet, in order to avoid false positive results.

Methods of Detection

Another aspect of the present invention provides methods of detecting the presence or absence of an analyte in a sample using the assay device of the present invention. In one embodiment of the present method, a collection slide containing the sample is placed into the docking area of an assay device, as shown in FIG. 4. Extraction buffer 512 is applied to the buffer or solvent orifice of the collection slide. The extraction buffer elutes the analyte of interest from the sample, if the analyte is present. Buffer applied to the buffer orifice flows through the cover pad and into the sample collection area containing the dried sample. The dried sample is rehydrated and a portion of the sample elutes out of the collection slide, through the eluent orifice. In one embodiment the buffer is pulled through the collection pad and onto the fluid transfer structure(s) by the fluid transfer structure(s) breaking the surface tension of the eluate. Excess buffer eluted from the collection slide is collected in the well surrounding the fluid transfer structure(s). Eluate on the fluid transfer structure(s) flows by gravity and capillary action into the application zone of the test strip, and then (by capillary action) to the downstream end of the test strip. As the eluate flows from the transfer structure(s) into the test strip, excess eluate held in the well may be transferred to the test strip, by the transfer structure(s).

As the eluate flows through the sample loading zone and reagent zone of the test strip, it dissolves reagents for conducting the assay present in the loading zone or reagent zone. In one embodiment these reagents are dried on the test strip. Reagents can also be included that condition the eluate for optimal detection, as described above. For example, if the assay is a sandwich format immunoassay reagents may include specific labeled binding molecules for the analyte, such as an antibody or fragment thereof. In one embodiment the specific binding molecule is a gold-labeled anti-hHb antibody or antibody fragment. If the analyte is present in the sample, the labeled specific binding molecule would capture the analyte and form a labeled, soluble complex, which is detected in the detection zone. The eluate continues to flow through the test strip to the detection zone, which contains a test line having specific binding molecules for the analyte. For example, the specific binding molecule can be an unlabeled antibody against the analyte, which binds at an epitope different from that of the labeling reagent. If the assay is a sandwich assay, the specific binding molecule in the test line captures the labeled antibody-analyte complex, and forms a visually detectable line indicating that the analyte is present in the sample. The test result therefore appears in the results window 128 located in the top portion of the housing.

In another embodiment the assay is a competitive format immunoassay. In this embodiment, the label zone or reagent zone of the test strip contains a labeled analog of the analyte, such as a gold-labeled hHb analog. If no analyte is present in the sample, the labeled analyte analog binds the antibody on the test line. Therefore a positive result on the test line indicates that no analyte is present in the sample. When analyte is present, it competes with the labeled analog to bind the antibody on the test line. As the concentration of analyte in the sample increases, the amount of analog that binds to the test line decreases. Therefore, a lighter line or no line indicates the presence of analyte in the sample.

A procedural control can also be included in the detection zone. The procedural control can be present as a line, and will always appear whether or not analyte is present in the sample. Absence of a positive result from the procedural control indicates an invalid assay.

In other embodiments the eluate is tested by means other than an immunoassay. For example, the analyte-containing eluate could be detecting using a chemical means, such as a Guaiac test or other chemical means.

Types of Samples and Analytes

A “sample” is any material to be tested for the presence, absence, or quantity of an analyte. In one embodiment the sample is a biological sample, such as a stool sample. But any type of sample can be assayed using the present invention, as long as it contains an analyte to be detected that can be solubilized and can be passed through the collection slide and into the assay device. The sample can be in many forms, such as solid, semi-solid or highly viscous materials, such as stool, soils, tissues, blood, bodily fluids, or macerated organs. The sample may also be an oral or vaginal swab.

A variety of analytes may be tested for using the present device. Examples of analytes that can be detected using the present invention include, but are not limited to, hemoglobin or other blood components, creatinine, bilirubin, nitrite, protein (nonspecific), hormones (e.g. human chorionic gonadotropin, luteinizing hormone, follicle stimulating hormone, etc.), leukocytes, sugars, heavy metals or toxins, bacterial components (e.g. proteins, sugars, or antigens specific to a particular type of bacteria, such as E. coli0157:H7, Staph. aureus, Salmonella sp., Salmonella typhii, Shigella, C. perfringens, Clostridium difficile, Campylobacter, Helicobacter pylori, L. monocytogenes, V. parahaemolyticus, Vibrio cholerae, or B. cereus), ova and parasites, and physical characteristics of the urine sample, such as pH and specific gravity. Any analyte can be detected for which a reliable assay can be designed. With reference to the present disclosure the person of ordinary skill in the art will realize a variety of antigens that can be detected using a variety of assay principles applicable in the invention.

Test Kits

A further aspect of the present invention provides kits containing one or more collection slides, and/or one or more assay devices of the present invention, and instructions for their use in carrying out an assay. The test kits can be packaged in a variety of formats, depending upon the needs of the user. In one embodiment the instructions provided with the kit are instructions for detecting the presence of hemoglobin in a stool sample.

In one embodiment, the kit contains three collection slides, three assay devices, three applicators, a desiccation mailing pouch having three sealable compartments, and instructions for collecting a sample, provided in a package. The package can be any suitable container. In various embodiments the package can be a box, a pouch, a bag, or can be simply a wrapping binding the items of the kit together.

In another embodiment the kits contain one or more collection slides and assay devices individually packaged in foil pouches, and one or more bottles of extraction buffer, and instructions, provided in a package. In another embodiment the kits contain three individually wrapped collection slides, extraction buffer for performing three tests, and instructions for use. At a health care facility where many tests would be conducted, the kit can contain many individually wrapped test devices, one or two large bottles of extraction buffer, and a single copy of the instructions.

A further embodiment provides a kit containing two “mini-kits,” wherein one mini-kit contains packaged together three collection slides, three applicators, a desiccant mail pouch and instructions for the patient explaining how to correctly collect the samples. The second mini-kit would contain, packaged together for the doctor, three test devices, extraction buffer sufficient to perform three tests and instructions for use.

### EXAMPLE 1

### Use of the Collection Slide and Assay Device for Analysis of hHb in Stool

Six collection slides of the invention were loaded with stool sample by smearing sample onto the sample collection pad. After drying, each slide was placed into the docking area of an assay device of the invention, as depicted in the Figures. By placing the collection slides into the docking area, the hinged side of the slide was inserted under the tang, and the slide pressed downward and snapped into place in the docking area, so that the eluent orifice of the collection slide was in fluid communication with the absorbent transfer material of the device.

Three drops (about 200 μl) of extraction buffer were then applied to the buffer orifice of the collection slide. In all cases, within 7-16 seconds, the buffer had flowed through the sample collection pad and out of the eluent orifice, and into the sample well. Within about 50 seconds, the buffer had flowed onto and through each of the test strips, and lines appeared at the control lines. The test strip had a test line with specific binding molecules for hHb, and a reagent zone with labeled antibodies for hHb.

The invention illustratively described herein may be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by various embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

1. A device for detecting an analyte in a sample, comprising:
a housing containing
a test element,
a docking area for receiving and engaging an external collection slide, the docking area comprising a sample receiving orifice having one or more fluid transfer structures comprised within the circumference of the sample receiving orifice; and
a results window for observing a test result.

2. The device of claim 1 wherein the sample receiving orifice is comprised in a well in the housing of the device.

3. The device of claim 1 wherein the one or more fluid transfer structures comprise a crossbar that projects above the plane of the docking area.

4. The device of claim 3 wherein the crossbar is positioned to be in fluid communication with an engaged collection slide.

5. The device of claim 1 wherein the docking area comprises one or more projections for securing the external sample collection slide in position above the sample receiving orifice.

6. The device of claim 5 wherein the one or more projections comprise one or more snap locks.

7. The device of claim 1 wherein the docking area is comprised as a depression in the housing and is at least partially circumscribed by a raised area of the housing.

8. The device of claim 1 wherein
the test element comprises a bibulous matrix having a sample application zone in fluid communication with the one or more fluid transfer structures;
a reagent zone comprising reagents for conducting an assay; and
a detection zone comprising a test line for visually detecting the presence or absence of the analyte at the test line.

9. The device of claim 8 wherein the test line further comprises a specific binding molecule for the analyte immobilized on the matrix.

10. The device of claim 9 wherein the specific binding molecule is an antibody.

11. The device of claim 9 wherein the specific binding molecule on the test line binds to human hemoglobin.

12. The device of claim 8 wherein the reagent zone comprises labeled specific binding molecule for the analyte.

13. The device of claim 1 wherein the analyte is human hemoglobin.

14. A method of detecting the presence or absence of an analyte in a sample contained in a sample collection slide, comprising:
placing a collection slide containing the sample into a docking area of a device for detecting analyte in a sample, wherein the device comprises:
a test element comprised within a housing;
a docking area for receiving a collection slide, the docking area comprising a sample receiving orifice having one or more fluid transfer structures comprised within the circumference of the sample receiving orifice; and
a results window for observing a test result; and
wherein the collection slide comprises
a first water resistant card having an eluent orifice;
a second water resistant card hingeably connected to the first card and having a solvent orifice, the collection slide having an open position and a closed position,
a sample collection surface present between the solvent and eluent orifices when the collection slide is in the closed position; and
applying an extraction buffer to the solvent orifice of the collection slide;
allowing the extraction buffer to pass through the sample area and through the sample receiving orifice and test element; and
observing a test result in the results window.

15. The method of claim 14 wherein the test element comprises,
a bibulous matrix comprising
a sample application zone in fluid communication with the one or more fluid transfer structures;
a reagent zone comprising reagents for conducting an assay; and
a detection zone comprising a test line for detecting the presence or absence of the analyte.

16. The method of claim 15 wherein the test line comprises specific binding molecules for the analyte.

17. The method of claim 15 wherein the test line contains reagents for conducting a chemical test.

18. The method of claim 14 wherein the analyte is human hemoglobin.

19. A kit for collecting a biological sample, comprising:
a device for detecting an analyte in a fluid comprising:
a housing containing
a test element,
a docking area for engaging a collection slide and comprising a sample receiving orifice having one or more fluid transfer structures comprised within the circumference of the sample receiving orifice;
a results window for observing a test result;
a sample collector;
an envelope for containing a loaded collection device; and
instructions for use; and
a collection slide comprising:
a first water resistant card having an inner surface and a eluent orifice;
a second water resistant card hingeably connected to the first card and having an inner surface and a solvent orifice, the collection slide having an open position and a closed position, wherein the solvent and eluent orifices are aligned when the collection slide is in the closed position; and
a sample collection area on the first water resistant card to which sample is applied for collection, present between the solvent and eluent orifices when the collection slide is in the closed position; and
a sample collector
provided in a package.

20. A kit according to claim 19 further comprising one or more bottles containing buffers for conducing an assay according to the instructions for use.
